(19) United States
(12) Patent Application Publication
(10) Pub. No.: US 2007/0056597 A1
(43) Pub. Date: Mar. 15, 2007

(54) Transplantation of human lipid cells and other tissue regenerative cells and methods of use ancillary to sports/entertainment/celebrity/performance employment contracts and policies insuring those contracts

(76) Inventors: Frederick J. Fitzsimmons, Wilmette, IL (US); Daniel D. Richard, Boynton Beach, FL (US)

Correspondence Address: COLEMAN SUDOL SAPONE, P.C., 714 COLORADO AVENUE, BRIDGE PORT, CT, 06605-1601, US

(21) Appl. No.: 11/515,608

(22) Filed: Sep. 5, 2006

Related U.S. Application Data
(60) Provisional application No. 60/714,277, filed on Sep. 6, 2005.

(51) Int. Cl. C12N 5/08

(52) U.S. Cl. 128/898

(57) ABSTRACT

The present invention relates to business methods for transplanting adult mesenchymal stem/progenitor and/or other regenerative cells ancillary to sports/entertainment/celebrity/performer medicine, athletic/entertainment/celebrity/performer performance contracts, including sports contracts or contract insurance policies in order to treat and/or prevent injuries to individuals, especially including athletes, musicians, singers, dancers, actors, entertainers, celebrities and/or performers who are subject to a risk of injury. The present methods may be used to reduce the time that an performance entertainer is injured and unable to participate in a particular physical activity such as a sport or other public entertainment setting and the likelihood that an injury to an performance entertainer will be of long duration and/or career ending. The present invention also relates to the expansion, programming and/or reprogramming of autologous lipid stem cells and the treatment of bones, tendons, ligaments, cartilage, other connective tissue, joints and fractures and other body tissue injuries of individuals especially performance entertainers in order to facilitate improved treatment, reducing the time for treating injury, the likelihood of debilitating injury and/or career-ending injury. The present invention is particularly applicable to the treatment of athletes, musicians, singers, dancers and other entertainers, celebrities and performers (“performance entertainers”) engaged in physical activities with lucrative contracts, especially long-term contracts which are insured by policies where injury is a potential threat to prowess, competence, performance and careers.

### RELATED APPLICATIONS

This application claims the benefit of priority of provisional application Ser. No. 60/714,277, filed Sep. 6, 2005, the entire contents of which application is incorporated by reference herein.

### FIELD OF THE INVENTION

The present invention relates to business methods for transplanting adult human lipid cells to include, but not limited to, mesenchymal stem and/or progenitor and/or other regenerative cells and other such related transplantable cells used for tissue regeneration, repair and/or rehabilitation purposes ancillary to sports/entertainment/celebrity/performance contracts or sports/entertainment/celebrity/performance contract insurance policies in order to treat and/or prevent injuries to individuals, especially including athletes and other entertainers/celebrities/performers exposed to injury, for example musicians/vocalists/dancers (e.g. pianists, violinists, singers, stage show and ballet dancers, etc.), actors/on-camera personalities/models (e.g. those that perform for the public and/or do their own stunts), sports figures (e.g. baseball, basketball, football, soccer, tennis, golf, etc.) (Collectively, these athletes and other entertainment, celebrity and performance professionals are referred to as “performance entertainers”).

The present methods may be used to reduce the time that a performance entertainer is injured and unable to participate in a particular sport or profession and the likelihood that an injury to that individual will be of long duration and/or career ending. The present invention also relates to the expansion of autologous lipid stem cells and the treatment of tendons, ligaments, cartilage, joints and fractures of individuals, including athletes, entertainers, celebrities, performers, and other such insured performance contract individuals as described herein in order to facilitate improved treatment, reducing the time for treating injury, the likelihood of debilitating injury and/or career-ending injury. The present invention is particularly applicable to the treatment of athletes, entertainers, celebrities, and performers, such as musicians, actors, entertainers, sports figures, public celebrity figures and other performers with lucrative contracts, especially long-term contracts which are insured by policies where injury is a potential threat to professional prowess and to careers, although all athletes, entertainers, celebrities and performers may be treated using the methods of the present invention.

The present methods can reduce the costs and increase the value of long-term professional sports, entertainment, celebrity, performance and/or sponsorship contracts, especially sports or sponsorship contracts for any person or entity having an interest in seeing the performance fulfill the terms of a contract, including corporate employers, entertainment companies or other entities, professional sports teams, sports owners, agents, contract representatives, commercial and/or other sponsors, and other contracting parties and even the professional athlete/entertainers, celebrities, performers themselves and reduce the insurance premiums paid on behalf of athletes, entertainers, celebrities and performers in order to insure payment in the event of a debilitating injury. The reduction in payments under these policies due to a sports/entertainment/celebrity/performer professional's (especially athlete's) return under terms of an employment contract is a further advantageous aspect of the present invention. Another aspect of the invention relates to the fact that arthroscopic, bone, ligament, tendon, cartilage, connective tissue and/or other tissue repair surgeries on athletes, entertainers, celebrities, and performers may be reduced or eliminated.

### BACKGROUND OF THE INVENTION

Professional athletes/entertainers/celebrities/performers are generally among the highest paid group of individuals due to enhanced value of talent in sports and other entertainment and public exposure fields. Their prolific earnings are placed at risk however by the fact that these individuals are constantly exposed to injuries including broken bones, torn cartilage, and shoulder separation, among numerous others, consequent to their profession. While injured athletes, entertainers, celebrities and performers in certain fields are paid during their period of disability/immobilization and recovery, in a number of instances the individual's salary is terminated or markedly reduced immediately upon injury.

### BRIEF DESCRIPTION OF THE INVENTION

The present invention relates to the use of lipid stem cells obtained from a performance entertainer, in particular, the buttocks, abdomen and/or other adipose tissue containing body sites, which can be expanded, programmed and/or reprogrammed and stored, or alternatively, stored and expanded, programmed and/or reprogrammed and used to promote healing of injuries to cartilage, tendon, ligament, other connective tissues, bone and other tissues of the performance entertainer, thus reducing the overall time that an individual will be unable to perform competitively and/or meet any other contractual performance specifications. It is preferable that at the beginning of a contract period, lipid tissue from the performance entertainer, preferably from the performance entertainer's buttocks, abdomen, and/or other adipose tissue containing body sites, will be taken and the lipid cells will be purified to produce pluripotent stem and/or progenitor and/or other regenerative cells which can be used to facilitate the rapid healing of damaged or broken bones, torn cartilage, tendons, ligaments, other connective tissues and/or other body tissues and other injuries to the bone, cartilage, tendons, ligaments and connective tissue and/or other body tissues of the performance entertainer.

The present invention is directed to a business method comprising: providing as a condition for employment as a professional athlete, entertainer, celebrity/performer, especially anyone required to perform physical tasks, especially athletic and/or other performance tasks under contract which compensates said performance entertainer, especially an athlete or performer in the event of an injury, a requirement that the performance entertainer, in the event of an injury to bone, a tendon, ligament, cartilage, connective tissue and/or other body tissues of said performance entertainer, will undergo a procedure to collect and isolate mesenchymal stem/progenitor and/or other regenerative cells from said performance entertainer; and apply said stem/progenitor and/or other regenerative cells and/or other such related regenerative cells, optionally in the presence of least one growth factor, to said injury to reduce the time that the performance entertainer is injured and unable to perform under contract.

In the present invention, the contract optionally may be insured by an insurance policy which is payable in the event the performance entertainer is injured while under contract.

In the method of the present invention, a clause is inserted into a contract with an performance entertainer which requires the athlete to undergo procedures to isolate a fat cell sample from the individual (preferably at least about 10-20 grams, preferably from the buttocks, abdomen and/or other adipose tissue containing body sites) and then purify and separate the lipid cells to obtain mesenchymal stem/progenitor and/or other regenerative cells which may be found in the sample. The purified/isolated mesenchymal stem/progenitor and/or other regenerative cells are useful for treating injuries to bones (fractures and the like), degenerative joints, injured tendons, ligaments, cartilage, and other connective tissues and/or other body tissues, thereby substantially reducing the time that an performance entertainer is unable to perform and concomitantly substantially reducing the amount and duration of payouts either by the entity having an interest in the fulfillment of the contract, whether that entity be a corporation, an entertainment company, a sports owner or employer, and/or any other contractor source, or the insurance company which has insured the contract, or even the insured performance entertainer.

The methods of the invention for treating physical injuries, especially athletic injuries hereunder are rather facile. By way of example, a sample of subcutaneous lipid cells (about 10-20 grams) is obtained by lipectomy from the patient in a region or tissue of the patient (legs, arms, abdomen, buttocks) having sufficient fat cells to produce approximately 10-20 grams or more of isolated fat cells, preferably from the patient's buttocks, then the sample is processed to remove and/or otherwise separate mature adipose tissue by processing and purifying the remaining undifferentiated mesenchymal stem cells/progenitor and/or other regenerative and endothelial precursor cells using specific growth factors, especially including connective tissue growth factors, such as CTGF, CTGF-2, CTGF-3 and CTGF-4 as disclosed, for example in U.S. patent application publication No. 2003/0180891 and U.S. Pat. Nos. 5,408,040, 6,358,741 and 6,534,630, (which patents are incorporated by reference herein) or other growth factors as described herein, optionally in a cellular nutrient medium which is preferably at least a minimum essential medium.

Once obtained and purified, these pluripotent stem/progenitor and/or other regenerative cells may be used directly to treat a patient, or stored for potential future use. In addition, these cells may be expanded, programmed and/or reprogrammed before treatment in the patient. In this method, the lipid sample may be used directly, the sample may be purified and the resulting purified stem/progenitor and/or other regenerative cells used directly or the lipid cells and/or the purified stem/progenitor and/or other regenerative cells may be grown for a period sufficient to expand, program and/or reprogram the stem/progenitor and/or other regenerative cells which are obtained from the lipectomy by growing the cells for at least one growth cycle, preferably several growth cycles, to obtain a population of mesenchymal stem/progenitor and/or other regenerative cells which may be used to treat athletic injuries. At any step along the process, tissue samples or purified cells, including expanded, programmed and/or reprogrammed cells may be cryopreserved.

The sample of mesenchymal stem/progenitor and/or other regenerative cells may be used directly by injecting the cells into the wounded area of the patient (performance entertainer) in proximity to or directly into the damaged bone, cartilage, joint, tendon, ligament, other connective tissue, and/or other body tissues so that the damaged tissue will be exposed to the stem cells and/or other regenerative cells obtained from the lipectomy, as well as the optional medium and/or growth factor which will greatly reduce the time and enhance the effectiveness of the healing process. In preferred aspects of the invention, the cells, which preferably include connective tissue growth factor and/or minimum essential medium, is injected directly into the injured site to be treated: an affected joint in the case of a degenerative joint disease, a bone break, a fracture line in the case of a fracture, to sites (preferably, multiple sites) into the lesion in tendons and ligaments, or sites of focal cartilage and/or other connective tissue and/or other body tissue damage. As the mesenchymal stem/progenitor and/or other regenerative cells which are used in the present invention are autologous because they are obtained from lipid tissue directly from the patient (performance entertainer) to be treated, there is no danger of rejection and the approach has a much greater likelihood of success than if heterologous/allogeneic (i.e., from an individual who is not the patient) cells were used.

Alternatively, lipid mesenchymal stem/progenitor and/or other regenerative adult stem cells from the performance entertainer may be used to expand, program and/or reprogram embryonic stem cells or stem cells isolated from umbilical cords, umbilical blood, placental membranes and/or placental blood, amniotic tissue and/or fluid, bone marrow, adult peripheral blood, and adult stem and/or other regenerative cells from any other adult source. In this aspect of the present invention, human adult stem cells from any adult source, or alternatively, embryonic or neonatal stem cells, including those obtained from otherwise discarded umbilical cords or placental tissue or alternatively, umbilical cord or placental blood which is obtained after and as a consequence of the delivery of a baby, may be reprogrammed to produce cells which may be used in therapeutic procedures which are autologous. The resulting stem cells, which have characteristics consistent with autologous embryonic stem cells, may develop and differentiate into any type of cell useful for treatment in this invention, especially stem cells which can be used to repair injured connective tissue, including bone, ligaments, tendons, cartilage and/or other body tissues as described herein.

In additional aspects of the present invention, the reprogrammed stem cells which are reprogrammed from embryonic stem cells or the umbilical cord or placental tissue itself or the blood of the umbilical cord or placenta, or amnion tissue and/or fluid, and adult stem cells from any other adult source are referred to as “reprogrammed” stem cells. These stem cells, due to the reprogramming, are autologous to the source cells, in particular the performance entertainer. In this aspect, as a consequence of reprogramming that simply creates reprogrammed stem and/or other regenerative cells from those original stem cells through exposure to a cell medium which is consistent with the makeup of the cytosol from embryonic stem cells, or alternatively, by fusing genetic material from an adult stem cell (or other adult cell) with a denucleated stem cell obtained from an embryo, umbilical cord, placental tissue or umbilical or placental blood, amnion tissue and/or fluid, or adult stem and/or other regenerative cells from any other adult source to produce a reprogrammed stem cell. The resulting reprogrammed stem cells are autologous to the host from which the reprogramming nuclear material came from and have similar characteristics with embryonic stem cells and are thus more readily differentiated into numerous other cells for use in the performance entertainer. An exciting feature of these reprogrammed stem cells is the fact that these stem cells have characteristics consistent with embryonic stem cells, can be developed or differentiated into virtually any other cell used in treatment and in the case of the use of the umbilical cord, placental tissue, or umbilical cord blood or placental blood, amnion tissue and/fluid, and adult stem and/or other regenerative cells from any other adult source this result can be obtained without the moral, legal or ethical dilemmas posed by the use of traditional embryonic stem cells.

### DETAILED DESCRIPTION OF THE INVENTION

The following terms are used throughout the present specification to assist in describing the instant invention. All terms used within the present specification have the common meaning which is known to those of ordinary skill in the art when used in the context of describing the present invention.

The term “patient” or “subject” is used throughout the specification to describe a human patient, preferably an athlete/entertainer/celebrity/performer, especially an athlete, to whom treatment, including prophylactic treatment, with the compositions according to the present invention, is provided. The term “athlete/entertainer/celebrity/performer” as used herein refers to an athlete, musician, singer, dancer, actor, entertainer, celebrity and/or performer, especially an individual who is subject to a risk of injury as a consequence of performance under a contract. Such term is also synonymous with the term “performer” and “performance entertainer”.

The term “lipid tissue” or “lipid sample” is used throughout the specification to refer to fat or adipose tissue samples obtained from an individual or individuals to be treated according to the methods of the present invention. The use of fat tissue, preferably from the buttocks, abdomen, arms, legs, or other body sites of a human as a source of stem/progenitor and/or other regenerative cells is advantageous because it can be obtained relatively easily and without trauma to the donor. Mesenchymal stem/progenitor and/or other regenerative cells can be isolated from lipid tissue and used directly in the present invention by administering to the injured performance entertainer, alone or in combination with other components as otherwise described herein or expanded, programmed and/or reprogrammed and used before or after a period of storage (cryopreservation). Mesenchymal stem/progenitor and/or other regenerative cells are preferably used for autologous transplantation directly into the wound to be treated, when and if needed. Lipid tissue is preferably obtained by removal of about 10-20 grams or more from the arms, legs, abdomen or buttocks of a subject/patient. These cells also may be used to reprogram embryonic stem cells or stem cells obtained from the umbilical cord, placenta or umbilical cord blood or placental blood from a neonate, or amnion tissue and/or fluid, bone marrow, adult peripheral blood and adult stem cells from any other adult source.

The term “stem cell” when used generically, refers to stem and/or progenitor and/or other regenerative cells taken from the performance entertainer which may be modified as otherwise described herein for use in therapy to treat the performer hereunder.

The term “effective amount” is used throughout the specification to describe concentrations or amounts of components such as tissue, cells, connective tissue growth factor, amount of minimum essential medium, components of cell media differentiation agents, and the like or other agents and/or components which, when used in context, are effective for producing an intended result including promoting the repair and healing of injured bone (for example a fracture), tendons, cartilage, ligaments, connective and other body tissues or treating a connective tissue disease or other connective tissue condition or for effecting a transplantation of those cells within the patient to be treated. Compositions according to the present invention may be used to effect repair of injury to connective tissue in the patient or subject from whom lipid tissue is taken. The result in a more rapid and efficient repair or healing of the bone or connective tissue injury to which the stem cells are applied (generally, by direct injection), whether that change is an improvement (such as healing or repair of a bone fracture, a rip or tear in cartilage, a tendon, ligament, other connective tissue and/or other body tissues) or reversing the degeneration of a connective tissue disease or condition, reducing a connective tissue deficit) or a complete cure or repair of the of the injury or condition treated.

The term “lipid cells” are cells having at least an indication of a lipid phenotype, and are obtained from sources in the body of a patient/subject, preferably the buttocks, abdomen, arms or legs of the patient/subject which will contain numerous mesenchymal stem/progenitor and/or other regenerative cells. Mesenchymal stem cells are cells obtained from fat tissue with the ability to proliferate, exhibit self-maintenance or renewal over the life-time of the organism and to generate clonally related connective tissue progeny, including chondrcytes and osteoblasts. Mesenchymal stem cells give rise to connective tissue cells and can replace a number of connective tissue cells in the adult.

The term “administration” or “administering” is used throughout the specification to describe the process by which mesenchymal stem/progenitor and/or other regenerative cells according to the present invention are delivered to a patient for treatment purposes. Mesenchymal stem/progenitor and/or other regenerative cells may be administered a number of ways but the preferred method is by direct injection into the injured site to be treated. Mesenchymal stem/progenitor and/or other regenerative cells may be administered in the form of whole lipid tissue, but preferably the mesenchymal stem/progenitor and/or other regenerative cells are first purified or isolated to a level approaching at least about 50% of a cellular sample (at least about 50% of the cells are stem/progenitor and/or other regenerative cells and no greater than about 50% of the cells are lipid cells). In other preferred aspects, the mesenchymal stem/progenitor and/or other regenerative cells are purified to a level approaching at least about 75+%, at least about 80+%, at least about 85+%, at least about 90+%, at least about 95+% or at least about 99+% of the cells in the sample to be injected into the patient. The compositions according to the present invention may be used without treatment with a growth factor, but preferably at least one growth factor selected from the group consisting of connective tissue growth factor (CTGF-1, CTGF-2, CTGF-3 or CTGF-4), transforming growth factor-beta (at least one of TGFβ1, 2 or 3) and bone morphogenetic factor (BMP 1, 2, 2A, 3, 4, 5, 6 & 7-BMP-6 is also known VGR-1) and preferably mixtures of these growth factors which will aid differentiation of the stem/progenitor and/or other regenerative cells into the specific types of cells (such as bone, ligament, tendon cartilage, joint, etc.) required to facilitate healing of the injury. Mesenchymal stem/progenitor and/or other regenerative cells may be administered alone or preferably in a cellular nutrient minimum or minimum essential medium as otherwise disclosed herein. Administration will often depend upon the injury or condition treated and is preferably via a parenteral route, for example, intravenously, by administration directly into the injured tissue.

The terms “grafting,” “engrafting” and “transplanting” and “graft” and “transplantation” are used throughout the specification synonymously to describe the process by which mesenchymal stem/progenitor and/or other regenerative cells are delivered to the injured site, such as affected joint areas in the case of degenerative joint diseases, the fracture line in fractures, into the lesions in tendons and ligaments and areas of focal damage in cartilage.

The term “essentially” is used to describe a method of purification or treatment which is at least 95+% effective, more preferably at least about 98%effective and even more preferably at least 99% effective. Thus, a method which “essentially” eliminates or heals an injury eliminates at least about 95+% of the injured cell population or tissue area, more preferably at least about 99% of the cell population. Preferred mesenchymal stem/progenitor and/or other regenerative cells according to the present invention, in certain preferred embodiments, are essentially free of lipid cells or hematopoietic cells (i.e., the CD34+ cellular component of a mononuclear cell fragment).

The term “non-tumorigenic” refers to the fact that the cells do not give rise to a neoplasm or tumor. Stem and/or progenitor and/or other regenerative cells for use in the present invention are generally free from neoplasia and cancer.

The term “nutrient cell medium”, “cell medium, “cell media” is used to describe a cellular growth medium in which the cells used in the present invention are growth, whether those cells are lipid cells which are purified to isolate a high concentration of mesenchymal stem/progenitor and/or other regenerative cells or the purified mesenchymal stme/progenitor and/or other regenerative cells themselves which are expanded or used to treat injury as otherwise described herein. Cellular media are well known in the art and preferably comprise at least a minimum essential medium plus optional agents such as growth factors, glucose, non-essential amino acids, insulin, transferrin and other agents well known in the art. In certain preferred embodiments at least one differentiation agent or growth factor is added to the cell media in which the mesenchymal stem/progenitor and/or other regenerative cells are grown in order to promote differentiation of certain cells within the mesenchymal and/or other regenerative fraction into bone cells and other connective tissue, such as ligaments, tendons, cartilage, other connective tissues and/or other body tissues, etc.

In one aspect of the present invention, lipid cells are grown in standard cellular media (preferably, at least a minimum essential medium supplemented with non-essential amino acids, glutamine, mercaptoethanol and fetal bovine serum (FBS)) are grown in a “stem/progenitor and/or other regenerative cell proliferation medium” (i.e., a medium which efficiently grows mesenchymal stem/progenitor and/or other regenerative cells) followed by growth in a “differentiation medium”, generally, a medium which is similar to the stem/progenitor and/or other regenerative cell proliferation medium with the exception that at least one at least one growth factor selected from the group consisting of connective tissue growth factor (CTGF-1, CTGF-2, CTGF-3 or CTGF-4), transforming growth factor-beta (at least one of TGFβ1, 2 or 3) and bone morphogenetic factor (BMP 1, 2, 2A, 3, 4, 5, 6 & 7-BMP-6 is also known VGR-1) and preferably mixtures of these growth factors is added to the medium and in certain cases, other growth factors are limited or removed). One of ordinary skill will readily recognize that any number of cellular media may be used to grow lipid cell fractions or mesenchymal stem/progenitor and/or other regenerative cell fractions of lipid tissue to provide appropriate proliferation media and/or differentiation media.

The term “separation” is used throughout the specification to describe the process by which mesenchymal stem/progenitor and/or other regenerative cells are isolated from a sample of lipid cells obtained from a patient/subject.

In this method of the present invention, lipid cells are removed from the arms, legs, abdomen and/or buttocks and/or other adipose tissue containing body sites of the patient/subject, with a preferred sample of approximately 10-20 grams or more of lipid cells obtained from the patient/subject.

The present invention is therefore directed to key employment entertainment and performance contracts, especially including athletic sports and performance contracts which guarantee payment even when an performance entertainer is injured and the insurance policies which insure against such injury of an performance entertainer. The invention herein contemplates offering alone or as a service ancillary to the contract or insurance policy, a method for enhancing the treatment of an injury to the performance entertainer, especially for example, a sports injury, especially an injury to the bones, joints, ligaments, tendons, cartilage, other connective tissues and/or other body tissues of an performance entertainer in order to shorten the duration of the injury to the performance entertainer, the result being that the payout to an injured performance entertainer, either through contract provisions guaranteeing compensation even when the performance entertainer is injured and/or under insurance policies insuring against injury, will be reduced, thus substantially reducing the costs of the contracting entertainment, sports, performance events company or other entity, sports team, agent, contract representative, commercial or other sponsor or insurance company to place high-priced athletic/entertainment/celebrity/performance talent under long-term contracts. In addition, because the likelihood of an injury, including an athletic injury being successfully treated is significantly increased, there will be substantially reduced risk in contracting with an performance entertainer for longer periods of time.

In association with the present method, under the terms of a prevailing contract and/or insurance policy, an performance entertainer is required/requested to submit to procedures to remove lipid tissue from that individual, purify and/or separate mesenchymal stem/progenitor and/or other regenerative cells from that lipid tissue and use those mesenchymal stem/progenitor and/or other regenerative cells in autologous procedures to treat the injury. Under the present method, the preparation of a lipid sample is obtained from the patient/subject, preferably from his or her buttocks, abdomen, and/or other adipose tissue containing body sites subcutaneously by lipectomy. Subsequently, mature adipose cells are removed and the remaining undifferentiated mesenchymal stem/progenitor and/or other regenerative cells and endothelial precursor cells are further processed, purified and exposed to specific growth factors as described above. The stem/progenitor and/or other regenerative cells may be used directly to treat injury, expanded, programmed and/or reprogrammed and used to treat injury, stored using cryopreservation or expanded, programmed and/or reprogrammed and then stored using cryopreservation techniques well known in the art. The actual cost of the present procedure is a small fraction of the cost of paying an injured athlete compensation under a typical contract for a duration of the typical injury.

The value of the present method to the entertainment and/or performance production entity having an interest in the performance entertainer's contract (which term can include for example, a cable or other television network, a sponsor of an event, a sports owner (an individual or other entity), insurance company or other guarantor of payment under the contract) in reducing costs associated with insuring such payment of an performance entertainer under contract in the event of injury is much greater than the cost of the procedure to treat an performance entertainer to reduce the time that the performance entertainer is unable to perform. The resulting reduction in time in which an performance entertainer is unable to perform due to injury substantially reduces the costs associated with granting or insuring any contract, especially long-term athletic/entertainer/celebrity/performer contracts. The benefit to the sports team owner, producer, sponsor of an event, network television, insurance company and/or other contracting parties in cost reduction is extension. The present invention therefore represents a unique business approach which is heretofore nonexistent in the sports/entertainment/celebrity/performer industry. Even an individual performance entertainer may participate.

1. A business method comprising:
providing as a condition for employment as a performance entertainer to perform physical tasks under contract which compensates said performance entertainer in the event of an injury, a requirement that the performance entertainer, either in anticipation of or in the event of an injury to body tissues of said performance entertainer, undergo a procedure to collect and isolate mesenchymal stem cells, progenitor cells or other mesenchymal regenerative cells from said performance entertainer; and apply said stem cells, progenitor cells, other regenerative cells or mixtures thereof, optionally in the presence of at least one growth factor, to said injury to reduce the time that the performance entertainer is injured and unable to perform under contract.

2. The business method set forth in claim 1 wherein said contract is at least one year long and said injury occurs to bone, a tendon, a ligament, cartilage or other connective tissues of said performance entertainer.

3. The business method set forth in claim 1 wherein said contract is at least 5 years long and said injury occurs to bone, a tendon, a ligament, cartilage or other connective tissues of said performance entertainer.

4. The business method set forth in claim 1 wherein said contract is at least 10 years long and said injury occurs to bone, a tendon, a ligament, cartilage or other connective tissues of said performance entertainer.

5. The method according to claim 1 wherein said stem cells, progenitor cells, regenerative cells or mixtures thereof are isolated from lipid cells taken from the buttocks, abdomen, arms, legs and/or other adipose tissue containing body sites of said performance entertainer.

6. The method according to claim 1 wherein said growth factor is selected from the group consisting of CTGF-1, CTGF-2, CTGF-3, CTGF-4, TGFβ1, TGFβ2 or TGFβ3, BMP 1, BMP2, BMP2A, BMP3, BMP4, BMP5, BMP6, BMP7 and mixtures thereof

7. The method according to claim 6 wherein said cells are grown in a nutrient cell medium prior to application to said injury.

8. The method according to claim 1 wherein said cells are applied to said injury in a nutrient cell medium in the presence of at least one growth factor.

9. The method according to claim 8 wherein said growth factor comprises CTGF-1, CTGF-2, CTGF-3 or CTGF-4.

10. A business method comprising:
providing an insurance policy for a contract for employment as an performance entertainer to perform physical tasks under contract which compensates said performance entertainer in the event of an injury which requires that the performance entertainer undergo a procedure to collect and isolate mesenchymal stem cells, progenitor cells, other regenerative cells or mixtures thereof from said performance entertainer; and apply said stem cells, progenitor cells, other regenerative cells or mixtures thereof, optionally in the presence of least one growth factor to said injury to reduce the time that the performance entertainer is injured and unable to perform under contract.

11. The business method according to claim 10 wherein said policy requires that the contract to be insured contains a provision requiring said performance entertainer to undergo said treatment.

12. The business method set forth in claim 10 wherein said contract is at least a year long and said injury occurs to bone, a tendon, a ligament, cartilage or other connective tissues of said performance entertainer.

13. The business method set forth in claim 10 wherein said contract is at least 5 years long and said injury occurs to bone, a tendon, a ligament, cartilage or other connective tissues of said performance entertainer.

14. The business method set forth in claim 10 wherein said contract is at least 10 years long and said injury occurs to bone, a tendon, a ligament, cartilage or other connective tissues of said performance entertainer.

15. The method according to claim 10 wherein said stem/progenitor and/or other regenerative cells are isolated from lipid cells taken from the buttocks, abdomen, arms, legs, or other adipose tissue containing body sites of said performance entertainer.

16. The method according to claim 10 wherein said growth factor is selected from the group consisting of CTGF-1, CTGF-2, CTGF-3, CTGF-4, TGFβ1, TGFβ2 or TGFβ3, BMP 1, BMP2, BMP2A, BMP3, BMP4, BMP5, BMP6, BMP7 and mixtures thereof

17. The method according to claim 16 wherein said cells are grown in a nutrient cell medium prior to application to said injury.

18. The method according to claim 10 wherein said cells are applied to said injury in a nutrient cell medium in the presence of at least one growth factor.

19. The method according to claim 18 wherein said growth factor comprises CTGF-1, CTGF-2, CTGF-3 or CTGF-4.

20. The method according to claim 14 wherein a beneficiary of said policy is an entertainment company, an owner of a sports team or franchise, a sports agent or a commercial sponsor.

21. A business method comprising:
providing as a condition for employment as a performance entertainer to perform physical tasks under contract which compensates said entertainer in the event of an injury, a requirement that the entertainer, in the event of an injury undergo a procedure to collect and isolate stem cells, progenitor cells, other regenerative cells or mixtures thereof from said entertainer, use said isolated cells to reprogram stem cells, progenitor cells, other regenerative cells or mixtures thereof from an embryo or from umbilical cord tissue, placental tissue, umbilical cord blood or placental blood, amnion tissue or fluid, bone marrow, adult peripheral blood, and adult stem cells from any other adult source and apply said reprogrammed stem cells, progenitor cells, regenerative cells or mixtures thereof, optionally in the presence of least one growth factor, to said injury to reduce the time that the entertainer is injured and unable to perform under contract.

22. The method according to claim 21 wherein said cells isolated from said performance entertainer are lipid cells.
